SEQUENCE LISTING (13 sequences)

SEQ ID NO: 1 - DNA, 12 bp, Artificial
  Synthetic oligonucleotide
cctcaaaactcc

SEQ ID NO: 2 - DNA, 12 bp, Artificial
  Synthetic oligonucleotide
ggagttttgagg

SEQ ID NO: 3 - DNA, 12 bp, Artificial
  Synthetic oligonucleotide
aacgcatgcgtt

SEQ ID NO: 4 - DNA, 10 bp, Artificial
  Synthetic oligonucleotide
cgcaattgcg

SEQ ID NO: 5 - DNA, 12 bp, Artificial
  Synthetic oligonucleotide
cgcgaattcgcg

SEQ ID NO: 6 - DNA, 14 bp, Artificial
  Synthetic oligonucleotide
acgcgaattcgcgt

SEQ ID NO: 7 - DNA, 16 bp, Artificial
  Synthetic oligonucleotide
acgcagaattctgcgt

SEQ ID NO: 8 - DNA, 20 bp, Artificial
  Synthetic oligonucleotide
aacgcaaagatctttgcgtt

SEQ ID NO: 9 - DNA, 13 bp, Artificial
  Synthetic oligonucleotide
cgcgaattcgcgt

SEQ ID NO: 10 - DNA, 14 bp, Artificial
  Synthetic oligonucleotide
cgcgaattcgcgtt

SEQ ID NO: 11 - DNA, 22 bp, Artificial
  Synthetic oligonucleotide
cgcgaattcgcgtttttttttt

SEQ ID NO: 12 - DNA, 12 bp, Artificial
  Synthetic oligonucleotide
cgcgaattcgcg

SEQ ID NO: 13 - DNA, 12 bp, Artificial
  Synthetic oligonucleotide
cgcgaattcgcg

(19) United States
(12) Patent Application Publication
(10) Pub. No.: US 2009/0263908 A9
(43) Pub. Date: Oct. 22, 2009

(54) Liquid crystal condensation of nucleic acid (na) complexes

(76) Inventors: Michi Nakata, Boulder, CO (US); Noel Clark, Boulder, CO (US); Tommaso Bellini, Milano (IT); Giuliano Zanchetta, Milano (IT)

Correspondence Address: LATHROP & GAGE LLP, 4845 PEARL EAST CIRCLE, SUITE 201, BOULDER, CO, 80301, US

(21) Appl. No.: 12/075,050

(22) Filed: Mar. 7, 2008

Related U.S. Application Data
(60) Provisional application No. 60/893,603, filed on Mar. 7, 2007.

(51) Int. Cl. G01N 33/00; C07H 21/00; C07H 21/04; C07H 1/00

(52) U.S. Cl. 436/94; 536/22.1; 536/23.1; 536/25.4

(57) ABSTRACT

Upon cooling solutions or melts of nucleic acids or other polymers in which the constituent molecules exhibit a varying tendency to form multi-strand complexes and thus liquid crystal phases, the complex-forming oligomers preferentially separate into the liquid crystal phase and condensing into liquid crystal domains. Because the tendency for liquid crystal phase formation correlates with the ability of the single strands to form duplexes, which in turn, depends on their degree of complementarity, separation of the liquid crystal domains thus constitutes a method for separating these molecules based upon their degree of complementarity.

### RELATED APPLICATIONS

This application claims priority of U.S. Provisional Application No. 60/893,603 filed Mar. 7, 2007, the content of which is hereby incorporated into this application by reference.

### STATEMENT AS TO RIGHTS TO INVENTIONS MADE UNDER FEDERALLY SPONSORED RESEARCH OR DEVELOPMENT

The invention was made with government support under National Science Foundation Grant number DMR0213918. The United States government has certain rights in the invention.

### BACKGROUND

1. Field of the Invention

The present disclosure relates to the structure and function of nucleic acid molecules. More particularly, it relates to the formation of liquid crystal phases by polynucleotide molecules and its practical applications.

2. Description of the Related Art

LC-phases of solutions of duplex DNA have been characterized by optical [1,2,3,4,5,6,7,8], x-ray [9], and magnetic resonance [10,11] methods for chain lengths N, ranging from mega-base-pair (bp) semiflexible polymers down to N ˜100 bp rigid rod-like segments of length L ˜33 nm, comparable to the B-DNA bend persistence length, Λₚ ˜50 nm [12]. These studies have revealed, at temperatures T below the duplex melting and conditions of low ionic strength where B-DNA chains are repulsive: isotropic (I); helixed chiral nematic (N*); uniaxial columnar (C_{U}); higher-ordered columnar (C₂); and crystal (X) phases vs. increasing DNA concentration.

### SUMMARY

The present instrumentalities advance the art by providing a new methodology for DNA purification by selectively separating single stranded DNA molecules that are more complementary to each other from molecules that are less complementary. The methodology disclosed herein may have broad application in DNA/RNA separation, identification, sequencing, or in determining the presence or absence of a particular mutation in a DNA/RNA molecule.

In this disclosure, the behavior of nucleic acid preparations is examined in which the constituent molecules exhibit a varying tendency for liquid crystal phase formation. In such solutions the fact that liquid crystal phases and isotropic or other phases spatially segregate constitute a means of separating the constituent molecules on the basis of their liquid crystal forming tendency. This phenomenon was discovered in studies of solutions of short DNA strands in which it was demonstrated that the tendency for liquid crystal phase formation was correlated with the ability of the single strands to form duplexes, which in turn, depends on their degree of complementarity. The separation of molecules on the basis of liquid crystal formation is thus a new way to separate them on the basis of their degree of complementarity.

It has been discovered that formation of nematic LC phases occurs in B-DNA duplexes of length 6 bp<N<20 bp (2<L<7 nm; 0.8<L/D<2.4). The isotropic-nematic transitions occur for N values an order of magnitude smaller than those predicted from φ_{IN}, precluding orientational ordering by the Onsager criterion. Additionally, robust columnar phases have been discovered for 6 bp<N<20 bp. These findings are especially surprising given results reported in previous studies [14, 15, 16, 17, 18, 19, 20].

The observation of nDNA LCs has led to a novel methods of condensation of complementary nDNA duplexes from a solution of complementary and noncomplementary oligomers. In nDNA mixtures the formation of complementary duplexes and their end-to-end assembly creates rigid anisotropic rods that not only order into LC phases but, because of both a depletion interaction and the incompatibility of rod-like and flexible polymers in solution, also become nearly immiscible with unpaired single stranded oligomers. By mixing a small fraction of complementary strands in nDNA mixtures, the complementary duplexes segregate almost completely into the LC domains that form upon cooling a solution from the I phase. These domains are made up almost entirely of complementary duplex stacks. Within these condensates, end-to-end assembly establishes a high concentration (several molar, estimated below) of contacting oligomer chain ends and terminal reactants, thus broadening the possibility for covalent linking of short complementary oligomers into longer ones [21], by inorganic catalysts, for example [22]. The fact that the LC ordering is found to depend sensitively on complementarity means that LC formation couples complementarity to end contacts among duplexes, and thus to the ability to grow and make more specific the interacting molecules. Thus, molecules that complementarily aggregate and assemble into larger units that phase separate have an advantage in a chemical race to grow in size and specificity over those that cannot phase separate. If the phase-separated domains are liquid crystal then the overall structure of the complementary assemblies generated will be governed by the LC geometry, as is actually the case for the linear structure of base-paired nucleotides. The intrinsic ease of sequence recombination in oligonucleotidic living polymerization provides a base for the synthesis of a large variety of sequences, and thus facilitate the exploration of a variety of structures, a requirement of prebiotic chemistry [21,22].

It is hereby disclosed a polynucleotide complex comprising two to three strands of polynucleotides, wherein said strands have less than 100 base pairs, said polynucleotide complex being capable of forming aggregates, and said aggregates being capable of forming a liquid crystal (LC) domain. In one aspect, these polynucleotide molecules may comprise at least two strands of polynucleotides, preferably 2-3 strands, wherein the at least two strands have from 6 to 20 continuous and complementary base pairs. Under the conditions described in the present disclosure, the two strands base-pair and form liquid crystal (LC) droplets (or domains) and may thus phase-separate from other molecules that remain in their original phase. In another aspect, the at least two strands of polynucleotides may have greater than 5 but less than 100 continuous and complementary base pairs and still possess the capability to phase-separate in an LC form. In another aspect, the strands have less than 30 base pairs, or alternatively, less than 11 base pairs.

In another aspect, it is disclosed a material comprising polynucleotide molecules, said material exhibiting at least one liquid crystal domain and at least one non-liquid crystal domain, wherein both the liquid crystal domain and the non-liquid crystal domain coexist and contain at least one polynucleotide molecule. In a preferred embodiment, the material is in the form of a solution or a melt.

The polynucleotide molecules described herein may thus be purified by taking advantage of their unique LC-forming capabilities. Such molecules may encode polypeptides that are useful in fields such as medicine, agriculture, among others. The polynucleotide molecules may be also administered to an animal or human being for therapeutic purposes. The spontaneous assembly and stacking of polynucleotides may also be employed to study or to simulate the evolution of ribonucleotides in prebiotic era.

The polynucleotide molecules of the present disclosure may be a DNA or RNA molecule. The formation of the liquid crystal phase by the complementary strands may facilitate separation of these DNA or RNA molecules from a mixture of DNA or RNA molecules. In a preferred embodiment, the mixture of DNA or RNA molecules exist in an aqueous phase, from which the LC-forming duplexes may be separated.

Thus, according to the present disclosure, a heterogeneous population of molecules in a solution of melt may be separated by formation of liquid crystal domains (or phases), if these molecules exhibit varying degrees of tendency for forming multi-molecular complexes in a liquid crystal phase. The separation may be effected by separating the molecules in the liquid crystal phase from molecules in the non-liquid crystal phase of the solution or melt. More specifically, certain complementary polynucleotide strands may be separated from a mixture of single-stranded polynucleotide molecules by a process comprising the steps of: (a) allowing some molecules to form a liquid crystal phase; and (b) separating said liquid crystal phase from the rest of said mixture of polynucleotide molecules in a non-liquid crystal phase.

It is also disclosed a method for separating polynucleotide molecules capable of forming a liquid crystal phase from a mixture of polynucleotide molecules wherein some molecules are capable of forming a liquid crystal phase and some molecules are not capable of forming a liquid crystal phase, comprising the steps of (a) allowing some molecules to form a liquid crystal phase; and (b) separating said liquid crystal phase from the rest of said mixture of polynucleotide molecules in a non-liquid crystal phase.

The disclosed method may apply to a mixture or a material in a variety of physical forms, including, for example, a solution or a melt. It is to be recognized that under certain conditions, some potentially complementary strands may fail to base pair with another strand and remain unpaired at the end of the process. Thus, the unpaired single strands may contain potentially complementary as well as noncomplementary strands.

The formation of liquid crystal domains as described in step (a) may be enabled by lowering the temperature and the separation of liquid crystal domains in step (b) may be enabled by sedimentation. Preferably, the sedimentation of liquid crystal domains may be carried out by centrifugation. More preferably, the steps (a) and (b) may be carried out simultaneously. The temperature for LC formation is typically below Tm, but as shown in the Examples, the optimal temperature also depends on the concentration of the polynucleotide.

The present disclosure also provides a method for identifying a target polynucleotide molecule within a population of molecules, which may include, but are not limited to polynucleotides, polypeptides or other molecules. The identification process may generally include the following steps: (a) exposing the population of polynucleotide molecules to a single-stranded probe molecule, said probe molecule being complementary to the target molecule; (b) forming a duplex by base-paring the probe molecule with the target molecule; (c) allowing the duplex to form a liquid crystal phase; and (d) detecting the existence of the liquid crystal phase. The detection step may include characterization of the liquid crystal using optical, x-ray and magnetic resonance methods (Ref 1-11).

### BRIEF DESCRIPTION OF THE DRAWINGS

FIG. 1: (a) Nano-length B DNA complementary duplexes can be idealized as hydrophilic cylinders with hydrophobic ends, with the hexamer (6 bp in length) illustrated here having a diameter comparable to length. While such objects individually are not anisotropic enough in steric shape to produce liquid crystal phases, the hydrophobic ends cause end-to-end sticking and thus the formation of much more anisotropic assemblies that can orientationally and positionally order into liquid crystal phases. The nematic (N*) phase is formed at lower concentration and the unixaial columnar (CU) at higher concentration. (b) Upon cooling a mixture of complementary (yellow/green) and noncomplementary (orange) single stranded nDNAs the complementary oligomers base pair to form duplexes which then assemble into chains as in (a), and phase separate to liquid crystal domains. The result is that the complementary DNA is condensed and into the liquid crystal drops and the terminal chain end pairs become highly concentrated.

FIG. 2: Optical textures of the LC phases of a series of solutions of nDNA of increasing length obtained by depolarized light microscopy. Samples thickness is in the range 4 μm<t<8 μm, between glass plates. Isotropic (I) regions are black. The chiral nematic (N*) phase appears as fluid birefringent domains when its helix pitch is a few microns or longer, or as a Grandjean texture with “oily streaks” exhibiting selective reflection from the helix (8 bp, 10 bp), when the pitch is shorter than ˜700 nm. The columnar C_{U} phase is identified by its smooth developable domains, a consequence of the uniaxial symmetry and splay expulsion of the columnar ordering. At yet higher DNA concentration the C₂ phase exhibits dendritic growth forms, indicative of lower symmetry, more solid-like ordering. The width of each image is 120 μm.

FIG. 3: Experimental c (DNA concentration, c, in units of mg solute/ml solution)—N (oligomer length) phase behavior of nano- and long DNA, along with the theoretical behavior from several models of interacting rod-like particles. (a) The red triangles and solid red curve bound the I-N* phase coexistence for long (N>100) DNA [7,10]. The solid red circles and red dotted line give the N*-C_{U} phase boundary of long DNA [6,9]. For N<20, the phase transitions are marked by red open symbols: triangles for the I-N*, circles for the N*-C_{U} and squares for the C_{U}-C₂. The concentration range of each phase is indicated by colored columns: magenta (I), cyan (N*), yellow (C_{U}). Phase boundaries for these transitions from models of hard rod systems are shown for two choices of the volume fraction cp axis, one with the DNA effective electrostatic diameter D=4.0 nm (black lines/labels), applicable at low c, and the other with the DNA chemical diameter D=2.4 nm [34] (orange lines/labels). The latter is applicable at high c (i.e. small N), as indicated by the increase, with decreasing N, in C_{NCU}, the N*-C_{U} transition concentration. The black (Onsager) line, φ_{IN} ˜4D/L for D=4.0 nm, accounts well for the longDNA I=N* data. The dashed black line locates the N*-C_{U} transition concentration at μ=0.55, which is independent of L/D, as predicted from several theories [14,30,31], The open green dots represent the spherical particles of Lu and Kindt [16] (L=D=4.0 nm), and the construction with the closed green dots gives the aggregate lengths <N>, at which the N phase appears in their simulations. These lengths match the Onsager line well, indicating that the aggregates behave effectively as hard rods, justifying the similar construction in (b). The thin blue lines give the corresponding N*-C_{U} and I-N phase boundaries for D=2.4 nm. (b) The c-N phase diagram of (a) but scaled with respect to c_{NCU}. This plot shows that the N* range is decreasing with decreasing N. The arrow construction gives an estimate of the number of base pairs in an aggregate necessary to generate an Onsager nematic.

FIG. 4: Temperature (T)—concentration (c)—length (N) phase behavior of the series of nDNAs of FIG. 2. The shaded regions, indicating where each phase is observed, have nearly vertical boundaries, established by the concentration. Also shown are the duplex unbinding temperatures in the N* and C_{U} concentration range (open green circles and squares, respectively) of the N=10 and N=16 oligos. The unbinding temperature increases with increasing c.

FIG. 5: Optical texture of a 10 bp nDNA/900 bp longDNA contact cell obtained by depolarized light microscopy. In this cell the relative concentration of the 10 bp and 900 bp DNA varies from left to right, and the overall DNA concentration, c, increases from bottom to top. The chiral nematic phase appears first from the I phase (black) with increasing c and continuously spans the full 10/900 concentration range, indicating that the 10 bp nematic is the same phase as the 900 bp nematic. Similar remarks apply to the C_{U} phase. In the 900 bp DNA the helix is visible (pitch p˜3 μm), decreasing with increasing N=10 concentration to exhibit selective reflection in the red at the N=10 end.

FIG. 6: DTLM optical images of a uniaxial columnar phase texture between glass plates and synchrotron microbeam x-ray diffraction patterns of selected 10 μm×10 μm monodomains. Optical microscopy simultaneous with the x-ray scattering enables probing monodomains of either planar aligned developable domains (purple area) with the columns parallel to the glass, showing an array of linear columns (blue lines) in the x-ray, or domains with optic axis normal to the glass (red area), showing the hexagonal column lattice (blue dots) in the x-ray. The uniaxial hexagonal columnar structure of the nDNA C_{U} phase is confirmed by this experiment.

FIG. 7: (a) In a 1:1 mixture of complementary, but different, nDNA oligomers duplexes form upon cooling below their unbinding temperature T_{U} and the LC phase appears below TLC via a first order phase transition, filling nearly the whole area with LC domains (c), in this case of the C_{U} phase. (b) With one of the oligomer species in excess, in this case B, the transition to the LC phase is marked by the appearance of isolated LC (C_{U}) domains that sequester all of the other complementary oligomer, in this case A, into LC droplets. (c) DTLM images of LC domains of a 1:1 A:B mixture of the CCTCAAAACTCC (A) (SEQ ID. No 1)+GGAGTTTTGAGG (B) (SEQ ID. No 2) 12mers. (d) DTLM images of LC domains of a 1:10) A:B mixture of the same pair. The area occupied by LC domains in the 1:10 case is consistent with an essentially complete condensation of A oligomers into the LC phase.

FIG. 8: Combined DTLM and DRLM optical texture of a solution of the N=10 self-complimentary oligomer with concentration increasing from top to bottom. The high birefringence region in the lower quarter is the uniaxial columnar (C_{U}) and the rest is chiral nematic (N*). The reflection colors show that the N* pitch increases with increasing concentration. The peak in the red selective back-reflection band at the N*-C_{U} interface is for incident light of wavelength λₘₐₓ=625 nm, yielding a maximum pitch for N=10 of p=λₘₐₓ/n≈400 nm.

### DETAILED DESCRIPTION

The term “liquid crystal domain(s)” refers to the liquid crystal (LC) droplet(s) that are formed by the duplexes of complementary strands. “Non-liquid crystal domain(s)” refers to the phases that are not by definition in liquid crystal state. A solution is a homogenous mixture of one or more substances. For purpose of this disclosure, the material containing a population of polynucleotide molecules may exist in the form of a solution before the formation of liquid crystal domains (or phases) within the material. Strictly speaking, once the liquid crystal domains are form, more than one phases will occur in the material, and the term solution no longer applies to such a multi-phase material. For purpose of this disclosure, the term “solution” may sometimes be used even after the formation of the liquid crystal domains. Such uses are for the sake of convenience and by no means indicate that the material is still in a homogeneous state after the formation of the liquid crystal domains. The terms “pair” and “base pair” are sometimes used interchangeably as a verb to refer to the interaction between complementary nucleotides commonly known in the art.

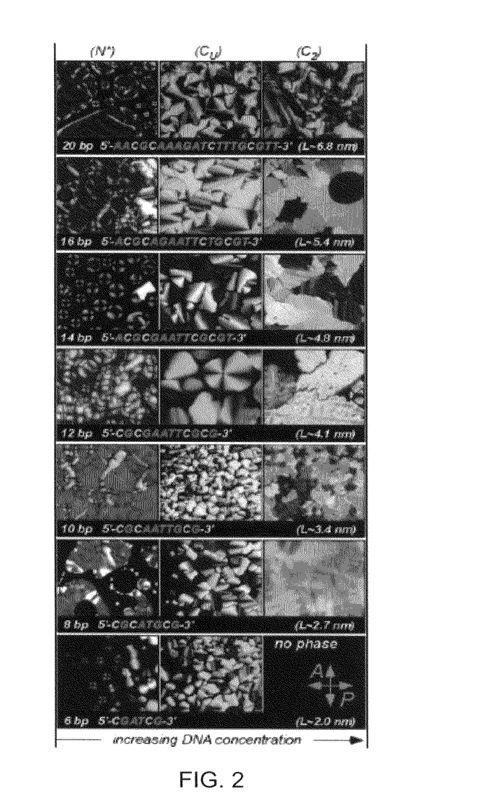

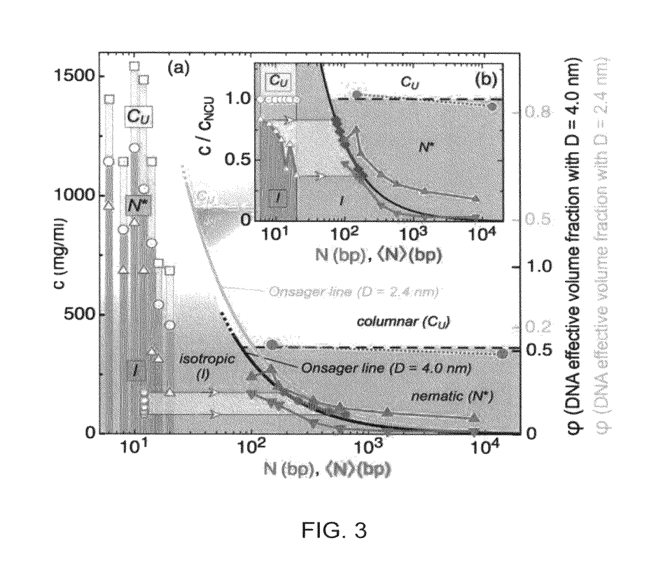

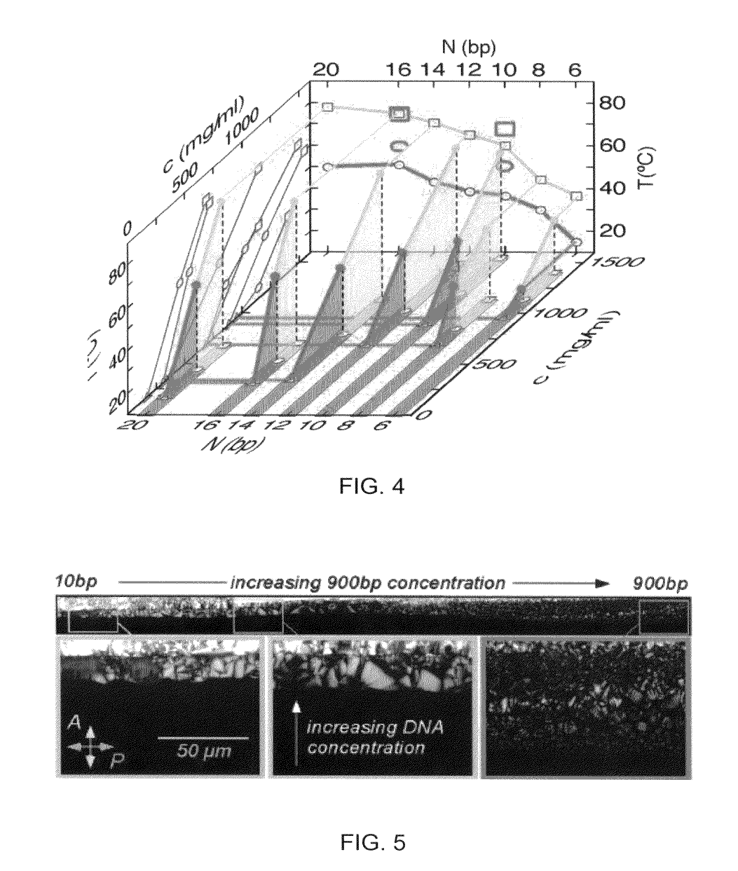

The series of self-complementary “palindromic” oligos for which data are here presented have sequences chosen so to avoid hairpin conformations and significant partial pairing. Among them is the extensively studied “Dickerson” 12mer, the B-DNA structure of which is maintained even in the lyophilized crystals, and is thus known in detail [23]. nDNA solutions in 4 μm<t<8 μm gaps between glass plates were studied by depolarized transmission light microscopy (DTLM) to probe optical textures; optical reflection interferometry (ORI) to measure refractive indices and thus DNA concentration c; and synchrotron microbeam x-ray diffraction (XRD) to probe local molecular organization. In spite of the challenges presented by the extremely small nDNA sample quantities available, these techniques nonetheless provided unambiguous evidence for helixed nematic (N*) and uniaxial columnar (C_{U}) liquid crystal phases in the nDNA solutions. At higher concentration, more ordered columnar (C₂) and crystal-like (X) phases were found, which have not yet been characterized in structure. FIG. 2 shows images of the typical textures, which were studied vs. temperature, T, and DNA concentration, c, (varying continuously across the sample area in the DTLM experiments). Optical texture and measurements of the local concentration (see Methods in the Supplementary Information) on the series of oligomers of length 6<N<20 in FIG. 2 enabled construction of the (N,T,c) phase diagram of FIGS. 3 and 4. In FIG. 3 we combine, on a c vs. N plot, phase boundaries measured for nDNA (open red symbols) with literature data on long DNA (full red symbols). Colored areas (for long DNA) and colored columns (for nDNA) indicate the concentration intervals where the I (magenta), N* (cyan), and C_{U} (yellow) phases are found. In the same figure we overplot the predictions from HSCYL (lines) and living polymer models (green dots), as detailed below. The nDNA solutions exhibited thermotropic mesomorphism: at sufficiently high temperature, T, the solutions melted to the optically isotropic liquid (I) phase. This is shown in FIG. 4, where we plot TLC, the largest T at which the N* and C_{U} phases are found. TLC grows with N and, for each oligomer, is larger for the C_{U} phase. At the concentration necessary for ordering into LC phases (300 mg/ml<c<1400 mg/ml), the counterion molarity m_{C} is in the range 2 M<m_{C}<10M. Thus, although prepared in pure water, the LC phases are obtained at effectively quite a large salt concentration. Addition of 0.2 M NaCl or 0.1 M MgCl₂ produced no significant variation of the phase behavior.

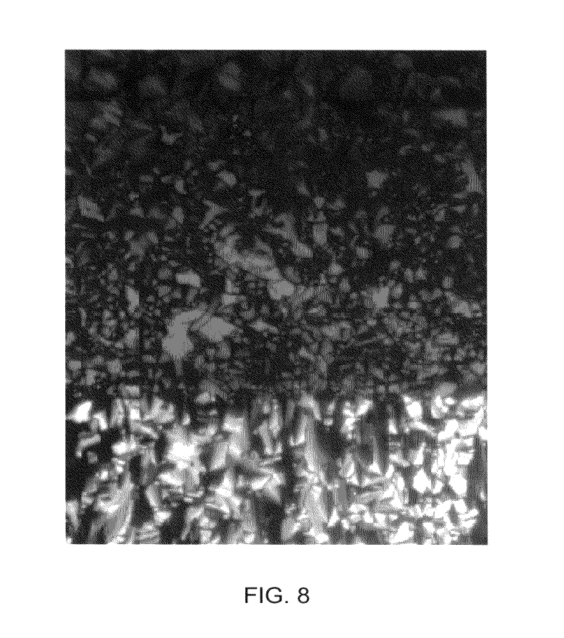

The N* phase appears with its chiral helix axis z either parallel (N*_{PAR}) or normal (N*_{NOR}) to the plates, forming either the focal conic (p<1 μm) or fingerprint (p>1 μm) textures in the PAR case (depending on the pitch, p, the distance required for a 2π director reorientation); and forming the Grandjean texture [24] in the NOR case. The pitch was observed to increase with increasing nDNA concentration (see Supplementary Information, FIG. 8). Also, as is clear from the selective reflection colors in the visible exhibited by the Grandjean textures, e.g., for N=8 bp in FIG. 2 and N=10 bp in FIG. 8, the pitch was found for several of the nDNA nematics to be p ˜0.6 μm, considerably smaller than the p ˜2.5 μm typically observed in N≧147 bp DNA. Birefringence measurements on the parallel textures using a variable wave-plate compensator enabled determination of Λn, the birefringence of the N* phase, revealing that the optical principal axis parallel to the helix axis has the higher refractive index, as is also the case for the N* phase in N≧147 bp DNA [5], i.e., that the nematic helix axis is parallel to the planes of the base pairs of the nDNA duplexes and normal to their double helix axes.

The DTLM observations clearly identify an N* phase in the nDNA solutions but leave open the question of its relation to the N* phase observed in longDNA. An approach widely used to identify unknown but possibly related LC phases is to fabricate a “contact” cell in which a quasi-linear concentration gradient in composition between the two compounds with the phases in question is established. If in DTLM observation a texture spans the concentration range without interruption by another phase, then the structural identity of the phases is established [24]. To compare the nDNA LC phases to those in longDNA, we prepared a contact cell with two distinct concentration gradients along orthogonal axes: a gradient between N=10 bp nDNA and N ˜900 bp DNA (S3h) along one axis, and a gradient in overall DNA concentration in the normal direction, as shown in FIG. 5. The N* phase (bordering the I phase) exhibits on the N=10 bp end, “focal-conic” N*_{PAR} and selective reflecting N*_{NOR} textures, characteristic of a nematic with p ˜600 nm. These textures evolve in a continuous way to the fingerprint texture of the N=900 bp end, indicating a single phase with p gradually increasing up to p ˜10 μm for N=900 bp. From this experiment, we conclude that the symmetry and structure of the nDNA N* phase is the same as the N* phase of longDNA: a helical winding along a helix axis (z) of a nematic director n(z) (in this case the optical polarization direction of low refractive index) in which n(z) is everywhere normal to z, and where n(z) gives the local molecular orientation of the DNA double helix axis.

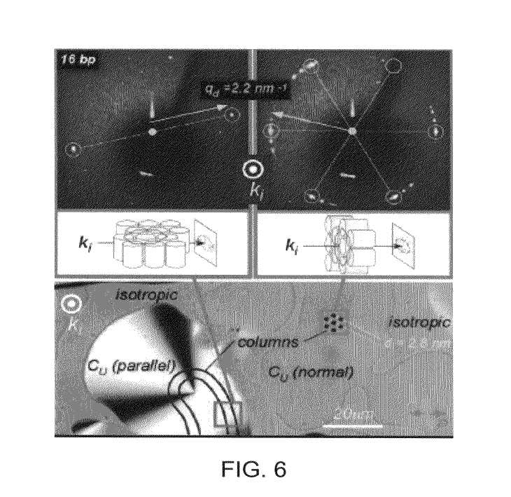

At higher nDNA concentration the uniaxial columnar phase (C_{U}) grew from the I upon cooling as a texture of two kinds of areas: (i) developable domains, as seen in FIGS. 2 & 6 [25], having birefringence substantially larger than that of the N* phase (Δn_{CU}˜2Λn_{N}*); and (ii) domains that are apparently isotropic, i.e., that have no in-plane birefringence (FIG. 6). Developable domains are clear indications of either fluid lamellar smectic or fluid columnar order with the layer normal or column axis respectively parallel to the glass plane. Variable wave plate study of the developable domain birefringence showed that the high refractive index direction is radial where the domains are circular, indicating that the DNA base pair planes are also radial and thus that the DNA double helix axes lie parallel to circles about the domain center (white rings in FIG. 6). Following the arguments of Livolant, formulated for longDNA, the structure is therefore columnar [5], with the double helix axes parallel to the columns, and in the birefringent domains the column axis is parallel to the glass plane (C_{UPAR}). The apparently isotropic domains (FIG. 6) have the column axes normal to the glass plane: they become birefringent upon tilting the sample, indicating that they are optically uniaxial with the uniaxis and thus the column axis normal to the glass (C_{UNOR}). The DTLM observations indicate unambiguously that the C_{U} phase is a uniaxial fluid columnar liquid crystal. This is also confirmed by measuring the specific birefringence for the C_{U} phase at the N* boundary (Δn˜0.025 for 350 mg/ml), which yields values in close agreement with those measured for the longDNA LC phases, once properly rescaled for concentration, thus supporting the notion that the packing of the nDNA phase is strictly analogous to that of the longDNA LC phases. This conclusion is also supported by the contact preparation of FIG. 5, wherein the C_{U} phase is also continuous along the 10 bp/900 bp concentration gradient.

Further structural characterization of the C_{U} phase was carried out using simultaneous DTLM and synchrotron-based microbeam x-ray diffraction. In these experiments the 10 μm×10 μm x-ray beam size was smaller than the texture domain size, so that area detection enabled efficient collection of the diffraction patterns of single 10 μm×10 μm×6 μm thick nDNA LC domains in a DLTM texture. FIG. 6 summarizes the results, showing diffraction from the principal C_{U} phase orientations: birefringent (C_{UPAR}) and isotropic (C_{UNOR}), confirming the nature of this phase as a hexagonal packing of uniaxial columns which lacks intracolumn positional correlation along the columns. These diffraction spots are resolution limited, as expected for the scattering from a C_{U} single domain [26].

The next higher concentration phase is another columnar phase (C₂) of lower symmetry than the C_{U}, which forms dendrite or tree-branch shaped domains of rectangular morphology and higher birefringence than the C_{U} phase, a result of the higher nDNA concentration. At yet higher concentration a solid-like phase (X) of lower birefringence appears, which might be either a crystal or an amorphous glassy phase. This phase sequence was observed for all of the 6 bp<N<20 bp palindromic nDNAs, with the exception that the C₂ phase does not show up for the N=6 bp oligomer.

The DLTM observations were made on cells with roughly linear gradients in c and in these cells each LC phase appeared as distinct domains upon cooling from the I phase only in a particular area of the cell, i.e., in a certain range of local nDNA concentration. That is, there were no thermally induced transitions observed between the different LC phases: they only melted to the I upon heating without transitions to the other LC phases, indicating that the phase boundaries in FIG. 4 (vertical dashed lines) are nearly parallel to the T axis. The duplex unbinding temperatures T_{U} for N=10 and 16 bp are also indicated in FIG. 4. The positions in c of these phase boundaries were determined by measuring the range of c in the various phases using simultaneous DTLM and ORI on cells made with high index glass. The N* phase was found to melt at the lowest T, with the C_{U} phase melting at a higher T directly to the I, and the C₂ phase melting at a yet higher T, also directly to the I and not through C_{U} or N* phases, which apparently require a different c. On cooling, the C₂ phase appears first directly from I, followed at lower T by I-C_{U} and I-N* transitions in cell regions of lower c. Transition temperatures measured on heating and cooling were not significantly different. The absence of temperature-induced transitions between lyotropic LC phases is not uncommon [27]. We probed the relationship of the LC phase transitions to the duplex unbinding transition by measuring the fluorescent emission of Ethidium Bromide (EtBr) mixed into the nDNA solution at a concentration of one molecule per duplex. Upon increasing T, as the nDNA duplexes unbind, the EtBr fluorescence is greatly reduced, enabling a determination of the fraction of paired strands [28]. We find that the nDNA unbinding is spread over a range of about 20° C., centered on a temperature T_{U}, defined as the 50% unpaired ratio, that grows with the density of nDNA. We find T_{U}˜T_{1-CU}, that is when c is large enough to give the C_{U} phase, then the DNA unbinding occurs along the LC melting transition to the I phase. At lower c we find that T_{U}˜T_{1-N}+10° C., implying that the N* phase is more readily disrupted by unpaired strands than the C_{U} phase.

As noted above, LC phases are found in nDNA under conditions where strictly repulsive hard rods of similar steric shape would be expected to be isotropic. Here we discuss the observed phase behavior of both longDNA and nDNA, plotted in FIG. 3, in the broader context of current models of LC formation in solutions of rod-shaped objects, including effects of flexibility, aggregation, and orientation. In the figure, experimental variables c (DNA concentration c in g solute/ml solution) and N are related to theoretical variables L and φ by considering the B-DNA double strands to be cylinders of mass density φ_{DNA}=1,800 mg/cm³ [29], of an effective diameter D ˜4.0 nm, chosen to fit the I-N and N-C_{U} phase boundaries measured for long DNA (longDNA) to models [7], as will be discussed below.

The longDNA I-N* phase boundary measured for 147 bp<N<8000 bp [7], and for N=100 [10] is shown in FIG. 3a by the red triangles, the two lines bounding the coexistence range. The phase boundary shifts up in c with decreasing L for L˜Λ_{P}˜50 nm, in reasonable agreement with the simple Onsager rigid-rod limit (black line and black labels on the right axis), if the effective double helix diameter is taken to be D_{eff}=4.0 nm to account for the electrostatic repulsion between chains [7]. Thus, while the longDNA I-N* phase boundary can be interpreted in terms of an electrostatically swollen DNA diameter, it is impossible to do so for the nDNA I-N* data on the basis of shape factors alone since any reasonable diameter yields axial ratios where there are no LC phases in any of the models.

The experimental N*-C_{U} phase boundary for longDNA (dotted red line and solid circles) can be obtained from the data of Rill, (N=147 bp [6]) and Podgornik (N=150 Kbp [9]). We note that for N>147 bp the concentration c_{NC} for the N*-C_{U} transition depends only weakly on N, as predicted by several of the models [14,15,30,31]. The choice of effective DNA diameter D_{eff}=4.0 nm yields an effective N*-C_{U} transition volume fraction of φ_{NCU}=0.55, close to the model predictions [14,30,31] (FIG. 3a). Note that the simple HSCYL model of rigid monodisperse particles [14] organizes into the smectic A (SmA) phase at high c, but changes to the C_{U} phase upon the introduction of either polydispersity in HSCYL length [30,31] or by molecular flexibility [32,33], as these effects reduce the free volume gain if the system were to organize into layers. In the limit of large L/D ratio, a length polydispersity above 20% is sufficient to eliminate the SmA phase, replaced by a hexagonal C_{U} phase for φ>φ_{NC}˜0.6 [30].

Among the nDNAs the LC phases appear in the N=20 oligomer duplexes at concentrations in the range of those of the longDNAs, in spite of lacking a L/D sufficiently large to enable LC ordering in the HSCYL model. Of particular interest to note in this regard is that the N=20 N*-C_{U} transition concentration (˜450 mg/ml) is nearly the same as that of the longDNA (C_{NC}˜370 mg/ml). Given the lack of dependence of c_{NC} on L for longDNA, this fact suggests that the phase behavior of the N=20 oligomers might be understandable on the basis of the HSCYL model, if their effective length L_{eff} was appropriately adjusted. In the simplest picture this adjustment assumes end-to-end aggregation into units of total length L_{eff}=<N> (base height) [aggregation of <N>/N oligomers] sufficient to increase the N=20 L_{eff} to contact the Onsager line, as shown in the construction in FIG. 3b. For the N=20 case this implies aggregates of length <N>˜200, i.e., consisting of ˜10 oligomer duplexes.

Justification for obtaining LC formation from such end-to-end aggregation can be found in model “living polymer”—type systems, where monomers with no steric anisotropy can reversibly aggregate into linear chains [15,16]. These chains have an exponential or broader distribution of lengths and thus are intrinsically polydisperse [15,16]. Computer simulations of the LC behavior of spheres, reversibly aggregating into linear chains [16,17], and those of flexible rods [32] show that for large bending rigidity the aggregate I-N transition occurs according to the Onsager prediction if the average aggregate length <L> is used in the Onsager model. In FIG. 3a we report the I-N phase boundary of aggregating spheres according to the results of Ref. 16 and show that the axial ratio of the aggregates, of average size <N>, matches well that expected for hard rods (green dots and construction). Furthermore, the unavoidable length polydispersity accompanying such aggregation replaces smectic phases by columnar. Thus, if we can assume that a similar criterion would apply to similarly aggregated rod-shaped particles, then the estimate described in the previous paragraph for LC formation in the N=20 case is obtained. At higher concentration the system of spheres aggregating in rigid chains, studied by Taylor and Herzfeld [15], exhibits a transition to the C_{U} phase. The N-C_{U} phase boundary is predicted to be at about φ_{NCU}˜0.5 when the sticking strength is large enough to yield aggregates of axial ratio L/D>5 (dashed black line in FIG. 3 [15]).

As the oligomer length is decreased the C_{U} phase and the nematic phase persist for duplexes as short as N=6, although the concentrations required to obtain these phases increase sharply. For N=6 the N-C_{U} transition is found at c=1,200 mg/ml, about two thirds of that of neat duplex DNA c_{DNA}=ρ_{DNA}=1,800 mg/ml [29]. Thus the LC phases of the oligomers of smallest N may be better viewed as being like thermotropic LC phases, rather than like those of colloidal particles.

A notable feature of the nDNA phase behavior is the presence of the N* phase even for the smallest nDNA studied. The model systems, ranging from flexible aggregate chains [33] to infinitely long repulsive chains [30] clearly show a requirement for adequate rigidity (Λₚ<˜10D) for the nematic to appear. For the shortest nDNAs, the systems are of sufficiently high concentration to behave like single component thermotropic LC-forming systems, where axial ratios L/D>˜5 are required to exhibit nematic order. The only possible scenario to account for the nDNA behaving as anisotropic particles with L/D sufficiently large is end-to-end aggregation of the duplexes into oligomer chains of rigidity sufficient to enable nematic ordering. Both the infinite length and aggregate flexible rod models show that in addition to suppressing the N phase: (i) increasing flexibility elevates the concentrations needed to get both the N and C_{U} phases, possibly accounting for the increasing concentration scale for decreasing N; and (ii) since flexibility suppresses LC ordering, estimates of aggregate length using the Onsager line or other rigid chain phase boundaries are lower limits since flexible rods would have to be longer to give the LC phases [30,33].

At this high concentration where LC phases are found for the shortest oligomers, the interaxial distance approaches the chemical diameter [9,23,34] where steric repulsion dominates the interchain interactions. Hence, with decreasing N and increasing concentration, the effective chain diameter evolves from D ˜40 Å to D ˜24 Å. This change shifts the model phase boundaries in FIG. 3a to higher concentration (black cφ scale-orange φ scale), accounting for the increased concentration necessary for LC phase formation. This effective diameter variation is scaled out in FIG. 3b, where the concentration axis is normalized by c_{NC}, assuming the DNA concentration at the N-C_{U} transition (c_{NC}) to correspond, for each oligomer, to an effective volume fraction φ_{NC}˜0.55 (the estimate of φ_{NC} obtained by averaging over the results from the various models discussed above [14,30,31]). That is, the D appropriate for a given oligomer is that which makes its NC_{U} transition occur at φ˜0.55, which yields D=4.0 nm from the longDNA N*-C_{U} data, comparable to that required to also fit the longDNA I-N* data. The resulting scaled phase diagram in FIG. 3b illustrates that the nematic range in the nDNAs is shrinking as N decreases, indicating that the Λₚ˜5D limit is being approached for small N. The scenario of end-to-end sticking may be further analyzed in the frame of FIG. 3b. Once the effective volume fraction is held by the strict requirement placed by the N*-C_{U} transition, the amount of linear aggregation can be evaluated by horizontally projecting the I-N* data points onto the expected I-N L/D for linear aggregation; we deduce a mean average chain length of about 8 monomers, independent of N.

From the estimate of the length of the nDNA duplex chains and the DNA concentration it is, in principle, possible to estimate the end-to-end stacking energy ΔE_{S} between the duplexes. Since the x-ray experiments of molecular spacing of Podgornik et al. [9] show that significant osmotic pressure is necessary to achieve the concentrations required for the nDNA LC phases, indicating a net repulsive interaction (electrostatic+steric) between the DNA duplexes, the energy ΔE_{S} represents the difference between a pair of duplexes having the position and orientation of lowest energy relative to their mean repulsive interaction. In the systems of fixed concentration studied here the net repulsion is balanced by the confining walls and ΔE_{S} represents the energy available to internally structure the phase. In order to add single duplex oligomers to a semi-rigid duplex chain ΔE_{S} must be large enough to overcome the loss of orientational entropy ΔS of each new member, requiring ΔE_{S}>TΔS˜k_{B}Tln(ΔΩ/4π), where ΔΩ is the orientational phase space of the fluctuations in relative orientation of the duplexes in the chain. If the aggregated duplexes are not free to reorient about the helix axis, then an estimate for ΔΩ is ΔΩ-πΔθ², where Δθ is the mean square fluctuation in the relative orientation of helix axes in an aggregate. If the aggregates are of rigidity comparable to that of longDNA, then Δθ˜0.1 radian and TΔS_{θ}˜6k_{B}T, to be considered a minimum possible fee energy requirement for aggregation, i.e., of imposing common orientation within an aggregate.

Estimates for ΔE_{S} can be obtained from various expressions proposed to describe the living polymer size distribution and mean aggregate length either in dilute [19,35] or in semi-dilute [16] solutions. The diversity of assumptions and approximations yields to estimates for the sticking energy ΔE_{S} ranging from 4 to 8 k_{B}T per contact, in addition to TΔS_{θ}, each of them, though, not showing significant N dependence (see Supplementary Information). More recent approaches consider reversible aggregation in high concentration system [36,37] and obtain corrections to the scaling laws expected for dilute system, without however enabling explicit energy estimates. Another approach to estimating ΔE_{S} is simply from the terminal structure of the DNA duplex itself, noting that the H-bonded base pairs form hydrophobic planar structures which can lower their interfacial energy with adjacent water by moving close enough to another to expel the intervening water (the basic origin of the DNA helicity). This leads to an estimate of ΔE_{S}˜25 cal/Å² mol, frequently used e.g. to roughly estimate the contact energy of biological macromolecules interacting because of hydrophobic pockets [38,39]. Since the area of a H-bonded pair of nitrogeneous bases is about 70 Å², we estimate a stacking energy ΔE_{S}˜6 k_{B}T, in line with living polymerization estimates. This value is about half of the base stacking energy expected for duplets of free Hbonded base pairs [40] and for the stacking of G-quartets [20], and about half of the enthalpic gain experimentally evaluated for the combination of pairing and stacking [41]. Our determination of ΔE_{S} is however larger than the base stacking free energy within DNA molecules [42], as expected because of the stacking entropic penalties contributing to the free energy and because of the higher constraints imposed by the backbone to the positional and orientational freedom of the stacked bases. In this respect, the nDNA system constitutes a test bench for base stacking computations.

A variety of additional experiments were performed to probe the dependence of the nDNA LC behavior on oligomer base pair sequence and chain termination. These included study of 12mers that were either palindromic, but with a sequence different from that of the Dickerson oligomer (AACGCATGCGTT) (SEQ ID. No 3), or mixtures of 12mers with different sequences that were complementary, e.g., CCTCAAAACTCC (SEQ ID. No 1)+GGAGTTTTGAGG (SEQ ID. No 2). Each of these exhibits the N and C_{U} phases with concentration ranges comparable to those of the Dickerson 12mer in FIG. 2. In an effort to influence the duplex end-to-end adhesion we carried out further experiments on the Dickerson 12mers (D12), but with added unpaired tails, either 1T, 2T or 10T groups added at the 3′ terminals. We found that this modification suppresses the LC phases with the exception of the C₂ phase, which is still observed in the case of D12 with 1 T and 2T groups. We interpret this result as indicating that dangling ends reduce end-to-end adhesion, in line with the base-stacking concept. By contrast, D12 duplexes phosphorylated on the 5′ end exhibited nearly the same phase behavior as that of the —OH terminated D12 duplexes described in FIGS. 2-5. Such duplexes, bearing one phosphor group per end, have the same composition as obtained by fracturing a long DNA duplex. This is obviously not true for solutions of duplexes with no phosphors at the ends and for solutions of duplexes phosphorylated at both 3′ and 5′ terminals. We tested also this last case with D12 duplexes phosphorylated on both ends, and found that the N* and C_{U} phases were suppressed and only the C₂ observed, as with the 1T and 2T terminations.

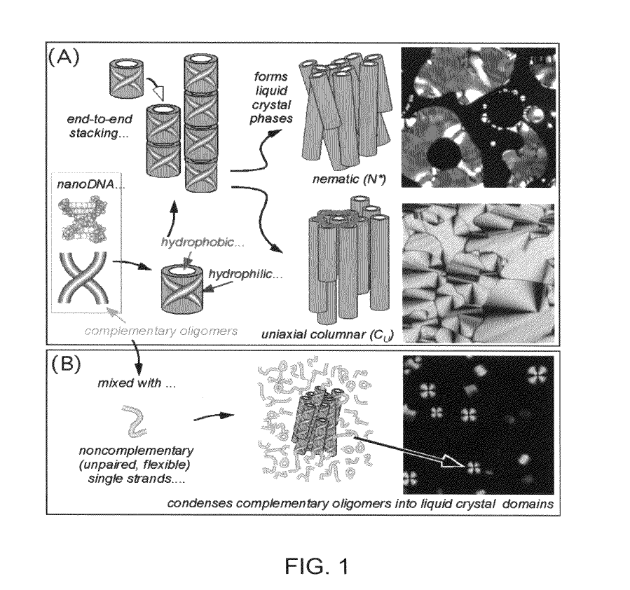

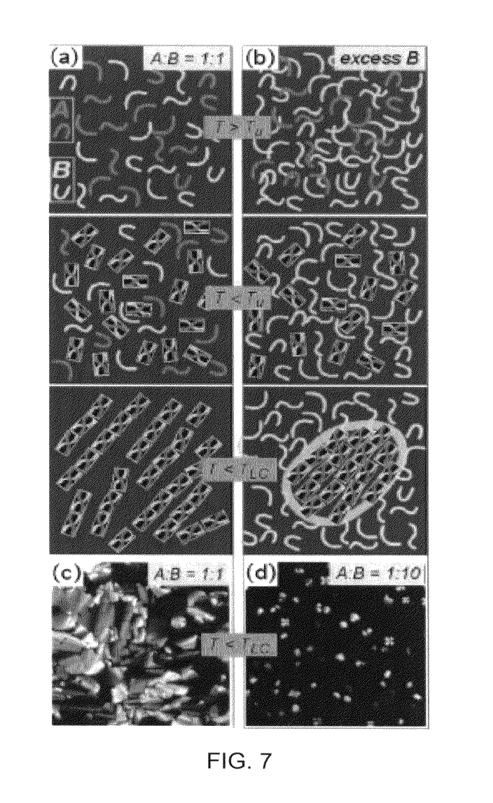

The aggregation reported here requires the formation of duplex DNA, which in turn requires a substantial degree of complementary pairing between the two chains. Thus, LC formation is a way to select complementary sequences from solutions of a variety of chains. In fact, we have found that the thermotropic first order transitions to nDNA LC phases reported above can be used to condense complementary duplex nDNA from a mixed solution of complementary and noncomplementary oligomers. This process is sketched in FIG. 7a,b and FIG. 1, and demonstrated in FIG. 7c,d for CCTCAAAACTCC (A) (SEQ ID. No 1)+GGAGTTTTGAGG (B) (SEQ ID. No 2) complementary 12mer mixtures. FIG. 7a sketches the typical solution structure vs. T reported above, here for a 1:1 A-B mixture, showing the solvated single-stranded oligomers at high temperature, duplex formation below the unbinding temperature T_{U}, and aggregation and LC formation below TLC. At 1:1, the transition temperature to a lower lying LC phase, typically nematic or C_{U}, depending on the concentration, nearly fills the sample with LC domains (FIG. 7c). The scenario in a solution where only a fraction of the oligomers are complementary, for example an A:B mixture with a large excess of B, is sketched in FIG. 7b. Here the duplex formation proceeds as for the 1:1, except that the excess B single strands are left unbound. FIG. 7d shows the result of cooling a 1:10 A:B mixture through the I-LC transition: the duplex units aggregate, and the solution separates into isotropic and LC phases. The dependence of the phase behavior on T and c, the area of the droplets, and the birefringence indicate that the duplexes are essentially insoluble in the isotropic and that the single stranded DNA is essentially insoluble in the LC. The I-LC phase separation condenses nearly all of the A-B duplexes, and thus localizes nearly all of the A oligomers into the LC drops. The situation that then emerges is not unlike the osmotic compression of DNA into LC phases obtained by adding a hydrophilic flexible polymer such as polyethylene glycol or dextran to a DNA solution [9].

The origin of this condensation is the contrast in rigidity of single and double stranded oligos. We mix flexible single stranded oligomers (Lₛₛ˜4 nm; Λₚₛₛ˜1-2 nm [43]) and rigid duplexes (L_{ds}˜4 nm; Λ_{pds}>>L_{ds}) which form semi-rigid aggregates (L_{ag}˜40 nm; Λ_{pag}>˜5 D˜20 nm.

Estimates provided by the various models for the duplex/single chain nDNA oligomers show strong segregation. For the c=400 mg/ml A:B=1:10 mixture of N=12 complementary oligomers shown in FIG. 7d, we find an LC/isotropic area ratio ˜7%, consistent with the ratio of duplex vs. single strand volumes, indicating strong segregation.

The LC ordering indicates end-to-end adhesion of the duplex nDNA into semi-rigid linear aggregates, which means that the terminal groups on neighboring oligomers are in close proximity, and thus that their effective concentration, c_{tLC}, is high. The most conservative estimate for ct assumes that the duplexes in a chain are uncorrelated with respect to their orientation about the chain axis, and thus that the terminal groups occupy a toroidal-shaped volume v_{tLC} about the chain axis. We estimate this volume to be v_{tLC}˜0.8 nm³, yielding c_{tLC}˜2 Moles/l. The end group concentration in the isotropic phase in FIG. 7d (c=400 mg/ml of N=12 oligomers), is c_{tISO}=0.006 Moles/l, yielding a significant end group concentration enhancement, c_{tLC}/c_{tISO}˜320, upon LC condensation. This spontaneous condensation of complementary nDNA into LC drops, when combined with the introduction into the drops of chemistry yielding the covalent linking of aggregated duplexes, favors a self assembly process capable of generating extended complementary DNA duplexes.

Thus, the present study provides a robust liquid crystal formation methods in a rather wide selection of complementary nDNAs that is absent in noncomplementary nDNA. In addition it demonstrates the link between complementarity and condensation of duplex nDNA, and the role of aggregate shape in establishing this link, i.e., in the generation of assemblies that can form liquid crystal phases. These results provide methods of generating complementarily H-bonded molecular assemblies. Specifically, H-bonded aggregates that are complementary selectively self-assemble so as to enable fluid phase separation. The consequent increased local concentration promotes further growth of the complementary molecular units, then a positive feedback cycle for enhanced selectivity and complementarity is created.

### EXAMPLES

The following examples are meant to illustrate certain embodiments of the prevent invention, and not to limit the scope of the invention.

### DNA Oligomers

nDNA oligomers (HPLC purified and ion free) of 6 to 20 base pairs in length were purchased from Bionexus Inc., Oakland (Calif., USA) and Primm srl (Milano, Italy) and obtained as lyophilized powder. To obtain liquid crystal phases nDNA were mixed with distilled deionized water. “LongDNA” was generated from DNA sodium salt from salmon tastes (Sigma-Aldrich D1626) dissolved into pure water and sonicated for 3 hours by a Branson sonicator (SONIFIER 250) to reduce the molecular weight [10] around 500˜900 bp (sample “S3h”). The nDNA and longDNA-H₂O solutions were loaded into 4 μm<t<8 μm thick, 1 mm wide channels between two glass plates separated by thin polymer film spacers. We used normal glass slides for the optical microscopy, high refractive index F2 glass (Schott AG, Mainz, Germany, n=1.62) for concentration measurements, and 50 μm thick float glass for x-ray microbeam experiments. The concentration was progressively increased by slow evaporation of water.

The experiments reported in FIGS. 1-6 were performed with the following oligonucleotides, here reported from 5′ to 3′ end: CGATCG (6 bp, Self-Complementary (SC)), CGCATGCG (8 bp, SC), CGCAATTGCG (SEQ ID. No 4) (10 bp, SC), CGCGAATTCGCG (SEQ ID. No 5) (12 bp “Dickerson dodecamer” (D12), SC), ACGCGAATTCGCGT (SEQ ID. No 6) (14 bp, SC), ACGCAGAATTCTGCGT (SEQ ID. No 7) (16 bp, SC), AACGCAAAGATCTTTGCGTT (SEQ ID. No 8) (20 bp, SC). Experiments were also performed with different other OH-terminated oligonucleotides: AACGCATGCGTT (SEQ ID. No 3) (12 bp, SC), CCTCAAAACTCC (SEQ ID. No 1) (12 bp), GGAGTTTTGAGG (SEQ ID. No 2) (12 bp), CGCGAATTCGCGT (SEQ ID. No 9) (13 bp, partially SC), CGCGAATTCGCGTT (SEQ ID. No 10) (14 bp, partially SC), CGCGAATTCGCGTTTTTTTT (SEQ ID. No 11) (22 bp, partially SC). We also investigated D12 samples pCGCGAATTCGCG (SEQ ID. No 12), phosphorylated at the 5′ terminal, and pCGCGAATTCGCGp (SEQ ID. No 13), phosphorylated at both ends.

### Microscopy

After loading the sample into the channel, one side of the channel was closed using epoxy glue to slow down the water evaporation rate. Water evaporating slower from one of the channel end induces gradients of DNA concentration, higher at the open end and lower at the closed end. Samples were thermally cycled several times to enhance the concentration gradient. Phase transitions were monitored via depolarized transmitted and reflected light microscopy (DTLM, DRLM) on a polarized optical microscope (Nikon, Eclipse E400 POL) as a function of cell temperature, controlled by a heat stage (Instec, STC200D).

To make a contact preparation of nDNA (10 bp) and poly-DNA S3h, two small droplets of each sample on a glass slide were covered by a cover slip, gently brought in touch and left diffusing one into the other for about one hour. Water was subsequently slowly dried from the edge of the cover slip (top side in FIG. 5) until liquid crystal phases appeared.

### Concentration Determination

DNA concentration was determined from refractive index n measured through microscope based interferometry. DNA solutions were loaded into a channel and local reflection spectra were measured by a S200 UV-VIS spectrometer (Oceanoptics, Dunedin, Fla.) in the range 450-750 nm, where the DNA absorption spectrum is nearly flat. The observed Fabry-Perot fringes enabled extracting the optical path length across the cells, and thus the local value of n. Interferometry measurements were performed at room temperature (20C). Concentrations were determined from n by assuming dn/dc=1.75 cm³/g, i.e. averaging over the estimates in literature [48,49,50,51], and a DNA density of 1.8 g/cm³. We estimate the accuracy of the method to be within 10% (an error of 0.01 in refractive index corresponds to an error of 0.05 g/cm³ in mass concentration).

### Microbeam X-Ray Diffraction

To investigate the structure of the liquid crystal phase, x-ray diffraction measurements were carried out using a micro focused synchrotron X-ray beam (20BM at the Advanced Photon Source, Argonne National Laboratory, USA). The cross section of the beam at the sample was approximately 10 μm.×10 μm. An X-ray energy of 16 keV was used in order to minimize absorption by the 50 μm thick glass slides confining the sample. X-ray scattering from the sample was detected by a 2-dimensional area detector Bruker AXS CCD. The liquid crystal domain structure and beam location on the sample were monitored using DTLM. The beamsize was small compared to the uniaxial columnar domain size.

### Estimate of Stacking Energy

From the estimate of the length of the nDNA duplex chains obtained by requiring end-toend aggregate whose length would match the Onsager line at the scaled experimental concentration (see FIG. 2b), it is, possible to estimate the end-to-end stacking energy ΔE_{S} between the duplexes. This is done by adopting various approximate models, as in the following.

Following Lu&Kindt [52], in turn basing their estimate on Cates [53], average number M of monomers in a chain depend on ε=ΔE_{S}/k_{B}T and on the nDNA volume fraction φ as

$$M = \frac{〈 N 〉}{N} = \frac{1}{2} + \frac{1}{2} \sqrt{1 + 4 {ϕ}^{ɛ + {k}_{1} ϕ}} \qquad ( 1 )$$

where k_{I}˜1.45 is a virial coefficient taking into account the steric repulsion between monomers.

Following Horowitz et al. [54], M is can instead be obtained as

$$M = \frac{2 ϕ {e}^{ɛ}}{\sqrt{1 + 4 ϕ {e}^{ɛ}} - 1} \qquad ( 2 )$$

Following Teixeira et al. [55], M can be obtained with either through Eq. 1 in the case of “solid” chains, where no monomer interchange can take place, or else, if monomer exchanges within a chain are taken into consideration, by the combination of the two equations below

$$\begin{matrix}M = \frac{ϕ {e}^{ɛ}}{exp ( a {e}^{ɛ} - 1} & ( 3 a ) \\ ϕ = a exp ( a {e}^{ɛ} ) & ( 3 b )\end{matrix}$$

When incompressibility is added to the description, a different equation is obtained

$$M = \frac{{e}^{ɛ} {x}_{0} / {x}_{s} ( \sqrt{1 + 4 {e}^{ɛ} {x}_{0} / {x}_{s}} ( - 1 ) )}{1 + 2 {e}^{ɛ} {x}_{0} / {x}_{s} - \sqrt{1 + 4 {e}^{ɛ} {x}_{0} / {x}_{s}}} \qquad ( 4 )$$

where x₀ and sₛ are the molar fractions of solute (nDNA duplexes) and solvent (water) respectively.

The nDNA solution involves solute molecules much larger than the solvent molecules. Following Flory [56], the mixing entropy, and thus the chemical potential regulating the equilibrium, should be expressed in terms of volume fractions of solute and solvent, rather than in terms of number density. Following this route, and constraining the total volume of the system to be constant, we find the equilibrium mean length of the aggregates to be

$$M = \frac{{e}^{ɛ} ϕ}{ln ( 1 + {e}^{ɛ} ϕ )} \qquad ( 5 )$$

Equations 1-5 can be thus used to extract ΔE_{S} from the data, and from the construction in FIG. 1b. We obtain:

From Eq. 1: ΔE_{S}=5.0±0.5 k_{B}T

From Eq. 2: ΔE_{S}=4.3±0.5 k_{B}T

From Eqs. 3: ΔE_{S}=8.3±1.2 k_{B}T

From Eq. 4: ΔE_{S}=7.0±0.7 k_{B}T

From Eq. 5: ΔE_{S}=3.9±0.4 k_{B}T

Noteworthy, for every given model, the energy value extracted from the data does not show any dependence on N, except for the experimental uncertainties.

### LISTING OF REFERENCES

The following references along with patents and publication of patent applications cited throughout this disclosures are hereby incorporated by reference as if the full contents are reproduced herein:
1. Wilkins, M. H. F., Stokes, A. R. & Wilson H. R. Molecular structure of nucleic acids: molecular structure of deoxypentose nucleic acids. Nature 171, 738-740 (1953).
2. Lydon, J. E. The DNA double helix—the untold story. Liq. Crys. Today 12, 1-9 (2003).
3. Luzzati, V. & Nicolaieff, A. Small-angle x-ray diffraction study of gels of deoxyribonucleic acid and nucleoproteins. J. Molec. Biol. 1, 127-133 (1959).
4. Robinson, C. Liquid-crystalline structures in polypeptide solutions. Tetrahedron 13, 219234 (1961).
5. Livolant, F., Levelut, A. M., Doucet, J. & Benoit, J. P. The highly concentrated liquid crystalline phase of dna is columnar hexagonal. Nature 339, 724-726 (1989).
6. Rill, R. L., Strzelecka, T. E., Davidson, M. W. & van Winkle, D. H. Ordered Phases In Concentrated DNA Solutions. Physica A 176, 87-116, (1991).
7. Merchant, K. & Rill, R. L. DNA length and concentration dependencies of anisotropic phase transitions of DNA solutions. Biophys. J. 73, 3154-3163 (1997)
8. Livolant, F. & Leforestier, A. Condensed Phases Of DNA: Structures And Phase Transitions. Prog. Polym. Sci. 21, 1115-1164 (1996).
9. Podgornik, R., Strey, H. H. & Parsegian, V. A. Colloidal DNA, Curr. Opin. Colloid Interface Sci. 3, 534-539 (1998).
10. Brandes, R. & Kearns, D. R. Magnetic Ordering of DNA Liquid Crystals, Biochem. 25, 5890-5895 (1986).
11. Alam, T. M. & Drobny, G. Magnetic Ordering in Synthetic Oligonucleotides. A Deuterium Nuclear Magnetic Resonance Investigation. J. Chem. Phys. 92, 6840-6847 (1990).
12. Hagerman, P. Flexibility of DNA, Ann. Rev. Biophys. Biophys. Chem. 17, 265-286 (1988).
13. Onsager, L. The effects of shape on the interaction of colloidal particles. Ann. NY Acad. Sci. 51, 627-659 (1949).
14. Bolhuis, P. & Frenkel, D. Tracing the phase boundaries of hard spherocylinders. J. Chem. Phys. 106, 666-685 (1997).
15. Taylor, M. P. & Herzfeld, J. A model for nematic and columnar ordering in a selfassembling system. Langmuir 6, 911-915 (1990).
16. Lu X., & Kindt J. T. Monte Carlo simulation of the self-assembly and phase behavior of semiflexible equilibrium polymers. J. Chem. Phys. 120, 10328-10338 (2004).
17. Van der Schoot, P. & Cates, M. E. Growth, static light scattering, and spontaneous ordering of rod-like micelles. Langmuir 10, 670-679 (1994).
18. Lydon, J. E. Chromonic mesophases. Curr. Opin. Colloid Interface Sci. 8, 480-490 (2004).
19. Horowitz, V. R., Janowitz, L. A., Modic, A. L., Heiney, P. A. & Collings, P. J. Aggregation behavior and chromonic liquid crystal properties of an anionic monoazo dye. Phys. Rev. E 72, 041710 (2005).
20. Davis J. T. G-quartets 40 years later: from 5′-GMP to molecular biology and supramolecular chemistry. Angw. Chem. Int. Ed. 43, 668-698 (2004).
21. Joyce, G. F., The antiquity of RNA-based evolution, Nature 418, 214-221 (2002).
22. Ferris, J. P. & Ertem, G. Oligomerization of ribonucleotides on montmorillonite. Science 257, 1387-1389 (1992).
23. Wing, R. et al. Crystal structure analysis of a complete turn of B-DNA, Nature 287, 755758 (1980).
24. Dierking, I. Textures of Liquid Crystals (Wiley-VCH, Weinheim, 2003).
25. Kleman, M. Defects in liquid crystals, Rep. Prog. Phys., 52, 555-654 (1989).
26. Safinya, C. R., Liang, K. S., Varady, W. A., Clark, N. A. & Andersson, G. Synchrotron x-ray study of the orientational ordering D2-D1 structural phase transition of freely suspended discotic strands in HAT 11. Phys. Rev. Lett. 53, 1172-1175 (1984).
27. Petrov, A. G. Lyotropic State of Matter: Molecular Physics and Living Matter Physics (Taylor & Francis, London, 1998).
28. LePecq, J. B., Paoletti, C., A flourescent complex between ethidium bromide and nucleic acids: physical-chemical characterization, J. Mol. Biol. 27, 87-106 (1967).
29. Durchschlag, H. Specific volumes of biological macromolecules and some other molecules of biological interest. Chapter 3 in Thermodynamic Data for Biochemistry and Biotechnology (Hinz, H. J. editor, Springer-Verlag, New York, 1986).
30. Bates M. A. & Frenkel D. Influence of polydispersity of the phase behavior of colloidal liquid crystals: a Monte Carlo simulation study. J. Chem. Phys. 109, 6193-6199, (1998).
31. Bohle, A. M., Holyst, R. & Vilgis, T. Polydispersity and ordered phases in solutions of rodlike macromolecules. Phys. Rev. Lett. 76, 1396-1399 (1996).
32. Hentschke, R. & Herzfeld, J. Isotropic, nematic and columnar ordering in systems of persistent flexible hard rods, Phys. Rev. A 44, 1148-1155 (1991)
33. Selinger J. V. & Bruinsma R. F. Hexagonal and nematic phases of chains. II. Phase transitions. Phys. Rev. A 43, 2922-2931 (1991).
34. Mandelkern, M., Elias, J. G., Eden, D., and Crothers, D. M., The dimensions of DNA in solution, J. Mol. Biol. 152, 153-161 (1981).
35. Teixeira, P. I. C., Tavares, J. M. & Telo da Gama, M. M. The effect of dipolar forces on the structure and thermodynamics of classical fluids. J. Phys.: Condens. Matter 12, R411-R434 (2000).
36. Rouault, Y. Concentration-induced growth of polymerlike micelles. Phys. Rev. E 58, 6155-6157 (1998).
37. Greer, S. C. Reversible polymerization and aggregations. Annu. Rev. Phys. Chem. 53, 173-200 (2002).
38. Reynolds, J. A., Gilbert, D. B. & Tanford, C. Empirical correlation between hydrophobic free energy and aqueous cavity surface area. Proc. Nat. Acad. Sci. USA 71, 2925-2927 (1974)
39. Fersht, A. R. Structure and Mechanism in Protein Science. (W.H. Freeman & Co., New York, 1999)
40. Sponer, J. et al. Nature of base stacking: reference quantum-chemical stacking energies in ten unique B-DNA base-pair steps. Chem. Eur. J. 12, 2854-2865 (2006).
41. SantaLucia, J. & Hicks, D. The thermodynamics of DNA structural motifs. Annu. Rev. Biophys. Chem. 33, 415-440 (2004).
42. Kool, E. T., Hydrogen bonding, base stacking, and steric effects in DNA replication. Annu. Rev. Biophys. Biomol. Struct. 30, 1-22 (2001)
43. Tinland, B., Pluen, A., Strum, J. & Weill, G. Persistence length of single-stranded DNA. Macromolecules, 30, 5763-5765 (1997)
44. Flory, P. J., Statistical thermodynamics of mixtures of rodlike particles with random coils. Macromolecules. 6, 1138-1141 (1978).
45. Asakura, S. and Oosawa, F., On interaction between two bodies immersed in a solution of macromolecules. J. Chem. Phys., 22, 1255-1256 (1954).
46. Lekkerkerker, H. N. W. and Stroobants, A., Phase behaviour of rod-like colloid+flexible polymer mixtures. II Nuovo Cimento, 16D, 949-962 (1994).
47. Annunziata, O. et al. Effect of polyethylene glycol on the liquid-liquid phase transition in aqueous protein solutions. Proc. Natl. Acad. Sci. USA, 99, 14165-14170 (2002)
48. Jolly, D. & Eisenberg, H. Photon correlation spectroscopy, total intensity light scattering with laser radiation and hydrodynamic studies of a well fractioned DNA sample. Biopolymers 15, 61-95 (1976)
49. Petra Wissenburg, P., Odijk, T., Cirkel, P. & Mandel, M. Multimolecular aggregation in concentrated isotropic solutions of mononucleosomal DNA in 1 M sodium chloride. Macromolecules 27, 306-308 (1994)
50. Ferrari, M. E. & Bloomfield, V. A. Scattering and diffusion of mononucleosomal DNA: effects of counterion valence and salt and DNA concentration. Macromolecules 25, 5266-5276 (1992)
51. Goinga, H. T. & Pecora, R. Dynamics of low molecular weight DNA fragments in dilute and semidilute solutions. Macromolecules 24, 6128-6138 (1991)
52. Lu X., & Kindt J. T. Monte Carlo simulation of the self-assembly and phase behavior of semiflexible equilibrium polymers. J. Chem. Phys. 120, 10328-10338 (2004).
53. Van der Schoot, P. & Cates, M. E. Growth, static light scattering, and spontaneous ordering of rod-like micelles. Langmuir 10, 670-679 (1994).
54. Horowitz, V. R., Janowitz, L. A., Modic, A. L., Heiney, P. A. & Collings, P. J. Aggregation behavior and chromonic liquid crystal properties of an anionic monoazo dye. Phys. Rev. E 72, 041710 (2005).
55. Teixeira, P. I. C., Tavares, J. M. & Telo da Gama, M. M. The effect of dipolar forces on the structure and thermodynamics of classical fluids. J. Phys.: Condens. Matter 12, R411-R434 (2000).
56. Flory, P. Principles of Polymer Chemistry (Cornell University Press, Ithaca, N.Y., 1953)

We claim:

1. A material comprising polynucleotide molecules, said material exhibiting at least one liquid crystal domain and at least one non-liquid crystal domain, wherein both the liquid crystal domain and the non-liquid crystal domain coexist and contain at least one polynucleotide molecule.

2. The material of claim 1, wherein the material is in the form of a solution or a melt.

3. The material of claim 1, wherein the at least one polynucleotide molecule in the liquid crystal domain is less than 100 base pairs in length.

4. A material comprising polynucleotide molecules less than 100 base pairs in length, said material exhibiting at least one liquid crystal domain and at least one non-liquid crystal domain, wherein both the liquid crystal domain and the non-liquid crystal domain coexist and contain at least one polynucleotide molecule.

5. A solution or melt of polynucleotide molecules less than 100 base pairs in length, said solution or melt exhibiting at least one liquid crystal domain and at least one non-liquid crystal domain, wherein both the liquid crystal domain and the non-liquid crystal domain coexist and contain at least one polynucleotide molecule.

6. A polynucleotide complex comprising:
two to three strands of polynucleotides, wherein said strands have less than 100 base pairs, said polynucleotide complex being capable of forming aggregates, and said aggregates being capable of forming a liquid crystal (LC) domain.

7. The polynucleotide complex of claim 6, wherein the strands have less than 30 base pairs.

8. The polynucleotide complex of claim 6, wherein the strands have less than 11 base pairs.

9. The polynucleotide complex of claim 6, wherein the polynucleotide is a DNA molecule.

10. The polynucleotide complex of claim 6, wherein the polynucleotide is an RNA molecule.

11. A method for separating polynucleotide molecules capable of forming a liquid crystal phase from a mixture of polynucleotide molecules wherein some molecules are capable of forming a liquid crystal phase and some molecules are not capable of forming a liquid crystal phase, comprising the steps of:
(a) allowing some molecules to form a liquid crystal phase;
(b) separating said liquid crystal phase from the rest of said mixture of polynucleotide molecules in a non-liquid crystal phase.

12. The method of claim 11, wherein the mixture of polynucleotide molecules pre-exist in a solution before the separation.

13. The method of claim 11, wherein the molecules capable of forming liquid crystal phases substantially form complexes of two or three single strands, and said molecules that are not capable of forming liquid crystal domains remain unpaired single strands after the separation.

14. The method of claim 11, wherein the molecules capable of forming liquid crystal phases substantially form complexes of two or three single strands, said complexes stacking into linear aggregates, and said molecules that are not capable of forming liquid crystal domains remain unpaired single strands after the separation.

15. The method of claim 11 wherein the formation of liquid crystal domains in step (a) is enabled by lowering the temperature.

16. The method of claim 11 wherein the separation of liquid crystal domains in step (b) is enabled by sedimentation.

17. The method of claim 16 wherein said sedimentation of liquid crystal domains is enabled by centrifugation.

18. The method of claim 11 wherein the formation of liquid crystal domains in step (a) is enabled by lowering the temperature, the separation of liquid crystal domains in step (b) is enabled by centrifugation, and steps (a) and (b) are carried out simultaneously.

19. A method for identifying a target polynucleotide molecule within a population of polynucleotide molecules, comprising the steps of:
(a) exposing the population of polynucleotide molecules to selected single-stranded probe molecules, said probe molecules capable of forming liquid crystal domains when complexed with the target molecules of said populations;
(b) forming a liquid crystal domains by complexing the probe molecules with the target molecules;
(c) enabling the complexes to form a liquid crystal domains;
(d) detecting the existence of the liquid crystal domains.

20. A method of separating a heterogeneous populations of molecules in a solution of melt, said molecules exhibiting varying degrees of tendency for forming multi-molecular complexes in a liquid crystal phase, said separation being effected by separation of molecules in the liquid crystal phase from molecules in the non-liquid crystal phase of said solution or melt.

21. An oligonucleotide having a sequence selected from the group consisting of SEQ ID Nos. 1-13.
